(19) United States
(12) Patent Application Publication
(10) Pub. No.: US 2024/0173099 A1
(43) Pub. Date: May 30, 2024

(54) DENTAL IMPLANT SURGICAL DRILL GUIDE DEVICE

(71) Applicant: MEDIMECCA CO., LTD., Seoul (KR)

(72) Inventors: Jin LEE, Gwangmyeong-si (KR); Jin Hoon LEE, Seoul (KR)

(21) Appl. No.: 18/283,004

(22) PCT Filed: Nov. 19, 2021
(86) PCT No.: PCT/KR2021/017048

§ 371 Date: Sep. 20, 2023

(30) Foreign Application Priority Data
Apr. 6, 2021 (KR) 10-2021-0044585

(51) Int. Cl. A61C 1/08; A61C 8/00

(52) U.S. Cl. CPC A61C 1/084; A61C 8/0089

(57) ABSTRACT

The present invention provides a dental implant surgical drill guide device. The dental implant surgical drill guide device comprises: a body portion extending in a longitudinal direction in a cylindrical shape having a first diameter; a neck portion having a second diameter and extending in the longitudinal direction from an end of one side of the body portion; and an extension portion having a third diameter, extending in the longitudinal direction from an end of the other side of the body portion, and having a stepped structure with the body portion. The body portion, the neck portion, and the extension portion are formed integrally. The size of the third diameter is formed to be smaller than the size of the first diameter and larger than the size of the second diameter. The dental implant surgical drill guide device comprises a guard portion extending in the longitudinal direction in a cylindrical shape having a fourth diameter formed to be larger than the first to third diameters, the guard portion being formed to surround the body portion, the neck portion, and the extension portion by the outer surface thereof, having an empty space defined by the inner surface thereof, and guiding inflow and outflow of washing water. The guard portion comprises: a space portion spaced apart from outer surfaces of the body portion and the neck portion by a predetermined interval; and an engaging portion disposed on the lower end of the guard portion so as to protrude from the lower end to the inner surface and to be fitted/coupled to the stepped structure of the extension portion. The space portion comprises: a first space portion formed between the outer portion of the body portion and the inner surface of the guard portion such that the upper portion thereof is exposed; and a second space portion formed between the outer portion of the neck portion and the inner surface of the guard portion such that the lower portion thereof is blocked by fitting/coupling between the extension portion and the engaging portion. The width of the first space portion may be formed to be larger than the width of the second space portion, and the width of the engaging portion may be formed equal to the width of the extension portion.

### TECHNICAL FIELD

The present disclosure relates to a dental implant surgical drill guide device, and more particularly, to a dental implant surgical drill guide device that is capable of, in an alveolar bone drilling process using a dental implant surgical drill, guiding and supplying washing water to a drilling site by gravity and thus cooling the drilling site.

### BACKGROUND ART

Generally, an implant is a prosthesis capable of substituting for human tissue when the original human tissue is lost, but in dentistry, the word refers to an artificially implanted tooth. That is, implant placement is a surgery in which a fixture formed of a material that is not rejected by the human body such as titanium is implanted in an alveolar bone from which a tooth has been lost so that the fixture substitutes for the lost tooth root and an artificial tooth is fixed to the fixture to restore functions of the lost tooth.

Although peripheral teeth and bone are damaged with time in the case of a general prosthesis or denture, an implant may prevent damage to peripheral dental tissues and may be stably used because there is no secondary cause of dental caries. Also, because an implant has substantially the same structure as a natural tooth, the implant does not cause gum pain or a foreign body sensation and thus may be semi-permanently used when managed well.

Meanwhile, implant surgery is performed by using a drill to form a hole in an alveolar bone and placing a fixture in the hole, but surgery for drilling to form a hole and placing a fixture is different for each patient. This is because an implant placement position and the depth and direction at which the implant is placed should be determined in consideration of various factors such as a dental condition of a patient, a position of a tooth where implant surgery is required, a condition of an alveolar bone of a patient, and the like.

In this way, in the drilling task for forming a hole in an alveolar bone, there is a problem that accurately determining the depth and direction in the process of performing the task is quite difficult not only for a novice practitioner but even for an experienced practitioner.

Accordingly, in order to improve accuracy and convenience of dental implant surgery, an auxiliary tool referred to as a surgical guide is used to identify an accurate position and direction for performing the drilling task.

The surgical guide is custom-made for each patient in order to be mounted in an oral cavity and has a guide hole formed at a target position where implant surgery will be performed. Accordingly, a practitioner performs drilling in the corresponding guide hole, and here, a sleeve is mounted on a handpiece to be fitted into the guide hole of the surgical guide and guide the position of a drill and a drilling direction.

However, since the sleeve is fitted into the guide hole of the surgical guide while surrounding the drill, washing water supplied from the handpiece is blocked from being introduced into the drilling position through the guide hole.

The washing water may serve to prevent an excessive increase in the temperature of the drilling site in addition to removing foreign matter generated in the drilling process, but because the sleeve blocks the supply of the washing water, drilling has to be performed in multiple steps, and necrosis of the alveolar bone may occur due to a high temperature of a surgical site (refer to Korean Patent Registration No. 10-1631261).

### DISCLOSURE

### Technical Problem

The present disclosure is directed to providing a dental implant surgical drill guide device.

Objectives of the present disclosure are not limited to the above-mentioned objective, and other unmentioned objectives should be clearly understood by those of ordinary skill in the art from the description below.

### Technical Solution

One embodiment of the present disclosure provides a dental implant surgical drill guide device including: a body formed in a cylindrical shape having a first diameter and extending in a longitudinal direction; a neck having a second diameter and formed to extend in the longitudinal direction from one side end of the body; and an extension having a third diameter, formed to extend in the longitudinal direction from the other side end of the body, and having a stepped structure with the body, wherein the body, the neck, and the extension are integrally formed, a size of the third diameter is formed to be smaller than a size of the first diameter and larger than a size of the second diameter, the dental implant surgical drill guide device further includes a guard which is formed in a cylindrical shape having a fourth diameter formed to be larger than each of the first to third diameters, which is formed to extend in the longitudinal direction and surround the body, the neck, and the extension from outer side surfaces thereof, which has an empty space formed at an inner side surface, and which is configured to guide an inflow and outflow of washing water, the guard includes a space that is spaced a predetermined distance apart from the outer side surfaces of the body and the neck and a catching portion that is disposed at a lower end of the guard, protrudes from the lower end to the inner side surface, and is fitted and coupled to the stepped structure of the extension, the space includes a first space formed between an outer side of the body and the inner side surface of the guard and having an upper portion formed to be exposed and a second space formed between an outer side of the neck and the inner side surface of the guard and having a lower portion blocked due to the catching portion being fitted and coupled to the extension, a width of the first space is formed to be larger than a width of the second space, and a width of the catching portion is formed to be the same as a width of the extension.

In one embodiment of the present disclosure, the body may have one or more through-holes formed to have a predetermined shape and pass through a side surface of the body in a radial direction of the body, and the neck may include cutting grooves formed to be opened by cutting the neck in the longitudinal direction and cutting portions formed in plate shapes having the same width due to the cutting grooves and formed alternately with the cutting grooves.

In one embodiment of the present disclosure, the dental implant surgical drill guide device may be made of titanium and a titanium alloy, the dental implant surgical drill guide device may further include a connector formed between the body and the neck and having a predetermined length, and the connector may be formed to be inclined downward from the neck to the body.

Also, one embodiment of the present disclosure provides a dental implant surgical drill guide device in which a dental implant surgical drill and the dental implant surgical drill guide device are combined to drill a hole in an alveolar bone and form a drilling site, the dental implant surgical drill guide device including: the dental implant surgical drill guide device including a body which is formed in a cylindrical shape having a first diameter, extends in a longitudinal direction, and has one or more through-holes formed therein, a neck which has a second diameter smaller than the first diameter and is formed to extend in the longitudinal direction from one side end of the body, and an extension which has a third diameter smaller than the first diameter and larger than the second diameter and is formed to extend in the longitudinal direction from the other side end of the body; and a guard configured to surround an outer side surface of the dental implant surgical drill guide device and have an empty space formed therein, wherein the guard includes a first space formed between an outer side surface of the body and an inner side surface of the guard, a second space formed between an outer side surface of the neck and the inner side surface of the guard, and a catching portion coupled to the extension formed to have a step with the outer side surface of the body, a width of the first space is formed to be larger than a width of the second space, by gravity, washing water is delivered to the drilling site by moving to the first space and the second space and passing through the through-holes while leakage of the washing water is prevented by the guard, the dental implant surgical drill guide device includes a guard which is formed in a cylindrical shape having a fourth diameter formed to be larger than each of the first to third diameters, which is formed to extend in the longitudinal direction and surround the body, the neck, and the extension from outer side surfaces thereof, which has an empty space formed at an inner side surface, and which is configured to guide an inflow and outflow of washing water, the guard includes a space that is spaced a predetermined distance apart from the outer side surfaces of the body and the neck and a catching portion that is disposed at a lower end of the guard, protrudes from the lower end to the inner side surface, and is fitted and coupled to a stepped structure of the extension, the space includes a first space formed between an outer side of the body and the inner side surface of the guard and having an upper portion formed to be exposed and a second space formed between an outer side of the neck and the inner side surface of the guard and having a lower portion blocked due to the catching portion being fitted and coupled to the extension, a width of the first space is formed to be larger than a width of the second space, and a width of the catching portion is formed to be the same as a width of the extension.

Other details of the present disclosure are incorporated in the following detailed description and the accompanying drawings.

### Advantageous Effects

According to the present disclosure, in an alveolar bone drilling process using a dental implant surgical drill, by guiding and supplying washing water to a drilling site by gravity, the drilling site can be cooled, and necrosis of the alveolar bone caused by frictional heat can be prevented.

According to the present disclosure, by guiding an inflow and outflow of washing water and directly supplying the washing water to a drilling site, discharge of foreign matter generated in an alveolar bone drilling process using a dental implant surgical drill can be facilitated.

Advantageous effects of the present disclosure are not limited to the above-mentioned advantageous effects, and other unmentioned advantageous effects should be clearly understood by those of ordinary skill in the art from the description below.

### DESCRIPTION OF DRAWINGS

FIG. 1 is a lateral view for describing a dental implant surgical drill guide device according to one embodiment of the present disclosure.

FIG. 2 is a cross-sectional view for describing the dental implant surgical drill guide device illustrated in FIG. 1.

FIG. 3 is a view for describing cross-sections of the dental implant surgical drill guide device illustrated in FIG. 2.

FIG. 4 is a lateral view for describing a dental implant surgical drill guide device according to another embodiment of the present disclosure.

FIG. 5 is a cross-sectional view for describing the dental implant surgical drill guide device illustrated in FIG. 4.

FIG. 6 is a view illustrating a cross-section along line C′-C′ illustrated in FIG. 5.

FIG. 7 is a bottom view for describing the dental implant surgical drill guide device illustrated in FIG. 5.

FIG. 8 is a view for describing a coupling state between the dental implant surgical drill guide device illustrated in FIG. 4 and a dental implant surgical drill.

### MODES OF THE DISCLOSURE

Advantages and features of the present disclosure and methods of achieving the same will become apparent from the embodiments described in detail below with reference to the accompanying drawings. However, the present disclosure is not limited to the embodiments disclosed below and may be implemented in various different forms. The embodiments herein are provided to make the disclosure complete and to completely inform those of ordinary skill in the art to which the present disclosure pertains of the scope of the disclosure, and the present disclosure is defined only by the scope of claims.

Terms used herein are for describing the embodiments and are not intended to limit the present disclosure. In the specification, a singular expression includes a plural expression unless the context clearly indicates otherwise. “Comprises” and/or “comprising” used herein do not exclude the possibility of the presence or addition of one or more elements other than those mentioned. Throughout the specification, the same reference numerals refer to the same elements, and “and/or” includes each of mentioned elements and all combinations of one or more of the elements. “First,” “second,” and the like are used to describe various elements, but, of course, the elements are not limited by the terms. The terms are only used to distinguish one element from another element. Therefore, of course, a first element mentioned below may also be a second element within the technical spirit of the present disclosure.

Unless otherwise defined, all terms including technical or scientific terms used herein have the same meaning as commonly understood by one of ordinary skill in the art to which the present disclosure pertains. Terms, such as those defined in commonly used dictionaries, should not to be construed in an idealized or overly formal sense unless expressly so defined herein.

Hereinafter, embodiments of the present disclosure will be described in detail with reference to the accompanying drawings.

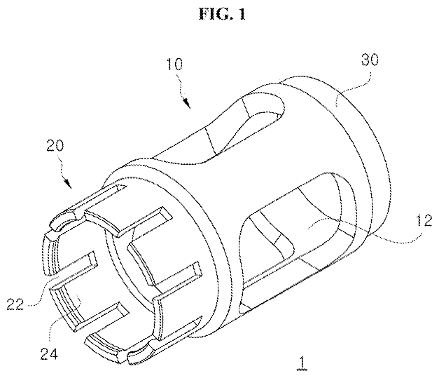

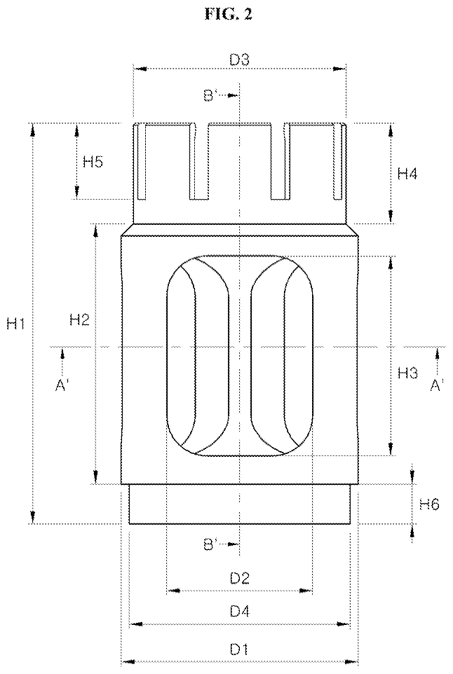

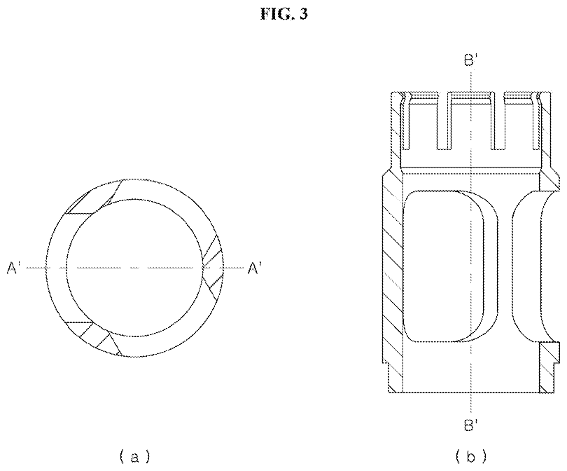

FIG. 1 is a lateral view for describing a dental implant surgical drill guide device according to one embodiment of the present disclosure, FIG. 2 is a cross-sectional view for describing the dental implant surgical drill guide device illustrated in FIG. 1, and FIG. 3 is a view for describing cross-sections of the dental implant surgical drill guide device illustrated in FIG. 2, wherein FIG. 3A is a view illustrating a cross-section along line A′-A′, and FIG. 3B is a view illustrating a cross-section along line B′-B′.

As illustrated in FIGS. 1 to 3, a dental implant surgical drill guide device 1 according to one embodiment of the present disclosure may include a body 10, a neck 20, and an extension 30. Here, the body 10, the neck 20, and the extension 30 may be integrally formed.

In the present embodiment, the dental implant surgical drill guide device 1 may be made of titanium, a titanium alloy, and the like that have excellent corrosion resistance and are stable even in human tissue. However, the present disclosure is not limited thereto, and the dental implant surgical drill guide device 1 may also be made of a metal or a metal alloy such as iron, chromium, nickel, stainless steel, a cobalt-based alloy, zirconium, niobium, tantalum, gold, or silver.

First, the body 10 may be formed to have a cylindrical shape having a predetermined diameter and extend in a longitudinal direction and may have a through-hole 12 formed in a side surface thereof.

The through-hole 12 may be provided as one or more through-holes 12 that pass through the side surface of the body 10 in a radial direction of the body 10. Here, the through-hole 12 is disclosed as having a quadrangular shape with rounded corners, but the present disclosure is not limited thereto.

In the present embodiment, the through-hole 12 is disclosed as being provided as three through-holes 12 that have the same shape as each other and are disposed to be spaced apart at equal intervals on the side surface of the body 10, but the present disclosure is not limited thereto.

For example, in the case in which an overall height H1 of the dental implant surgical drill guide device 1 is 10 mm in the longitudinal direction, a height H2 of the body 10 may be 6.5 mm, and a diameter D1 of the body 10 may be 5.9 mm. Here, a height H3 of the through-hole 12 may be 5 mm, and a diameter D2 of the through-hole 12 may be 3.63 mm.

By the through-hole 12 that passes through the side surface of the body 10 being provided in the body 10 as described above, foreign matter generated in the drilling process, for example, tissue in an oral cavity or fragments of an alveolar bone 52, may be smoothly discharged through the through-hole 12 together with washing water without being re-introduced into a drilling site. Accordingly, since the foreign matter is smoothly removed simultaneously as the washing water is supplied through the through-hole 12, necrosis of the alveolar bone 52 due to a temperature rise during the drilling process can be prevented.

The neck 20 extends in the longitudinal direction from one side end of the body 10 in a direction in which a dental implant surgical drill 50 is fitted and is integrally formed with the body 10. The neck 20 may include a plurality of cutting grooves 22 and cutting portions 24.

Specifically, a diameter D3 of the neck 20 may be formed to be smaller than the diameter D1 of the body 10, and the neck 20 may be formed in a hollow shape extending upward from the one side end of the body 10. Here, the neck 20 may be formed to extend from the body 10 by a connector having a predetermined length that is disposed between the body 10 and the neck 20 which are integrally formed. Here, the connector may be formed to be inclined downward from the neck 20 to the body 10.

For example, in the cross-sectional view of FIG. 2, in the case in which the overall height H1 of the dental implant surgical drill guide device 1 is 10 mm in the longitudinal direction, a height H4 of the neck 20 may be 2.5 mm, and the diameter D3 of the neck 20 may be 5.3 mm. That is, the diameter D3 of the neck 20 may be formed to be smaller than the diameter D1 of the body 10. Accordingly, the connector connecting the body 10 and the neck 20 may be formed to be inclined downward from the neck 20 to the body 10.

The cutting grooves 22 may be disposed to be uniformly spaced apart at predetermined intervals. That is, the cutting grooves 22 may be portions that are opened by cutting the neck 20 in the longitudinal direction so that the dental implant surgical drill 50 is easily fastened to the body 10.

In the present embodiment, the cutting grooves 22 each having a height H5 of 5 mm and a width of 0.5 mm may be formed by cutting the neck 20, but the present disclosure is not limited thereto.

The cutting portions 24 may be formed as plate shapes having the same width due to the plurality of cutting grooves 22, and the number of cutting portions 24 may be proportional to the number of cutting grooves 22. Here, the cutting portions 24 may be formed alternately with the cutting grooves 22.

For example, the plurality of cutting portions 24 may be disposed to be spaced apart by the width of the cutting groove 22, that is, 0.5 mm. Here, a height of the cutting portion 24 may be formed to be the same as the height H4 of the neck 20.

In the case in which the dental implant surgical drill 50 is rotated forward and fastened to the inside of the body 10 that has the neck 20 having the above-described configuration, with the rotation of the dental implant surgical drill 50, the cutting portions 24 of the neck 20 are closed due to the cutting grooves 22, and the dental implant surgical drill 50 can be fixed due to the neck 20 and prevented from being detached from the body 10. Therefore, dental implant surgery can be safely performed for both a practitioner performing the dental implant surgery and a patient receiving the dental implant surgery.

Meanwhile, in the case in which the dental implant surgical drill 50 fastened to the inside of the body 10 is rotated backward after the drilling process is completed, with the rotation of the dental implant surgical drill 50, the cutting portions 24 of the neck 20 are opened due to the cutting grooves 22, and the dental implant surgical drill 50 can be easily separated from the body 10.

The extension 30 is integrally formed with the body 10 and may be formed to extend in the longitudinal direction from the other side of the neck 20 and protrude from one side end of the body 10.

Here, a diameter D4 of the extension 30 may be formed to be smaller than the diameter D1 of the body 10. Also, the diameter D4 of the extension 30 may be formed to be larger than the diameter D3 of the neck 20. That is, an outer side surface of the extension 30 may be formed to have a step with an outer side surface of the body 10.

For example, in the cross-sectional view of FIG. 2, in the case in which the height H2 of the body 10 is 6.5 mm and the diameter D1 of the body 10 is 5.9 mm when the overall height H1 of the dental implant surgical drill guide device 1 is 10 mm in the longitudinal direction, a height H6 of the extension 30 may be 1 mm, and the diameter D4 of the extension 30 may be 5.5 mm. That is, the diameter D4 of the extension 30 may be formed to be 0.4 mm smaller than the diameter D1 of the body 10.

By the body 10 having the extension 30 that has the diameter D4 smaller than the diameter D1 of the body 10 as described above, in the case in which the dental implant surgical drill 50 is fastened to the inside of the body 10, the dental implant surgical drill 50 can be primarily fixed due to the neck 20 being closed and secondarily fixed by the extension 30 having the diameter D4 smaller than the diameter D1 of the body 10, and thus the dental implant surgical drill 50 can be further prevented from being detached from the body 10.

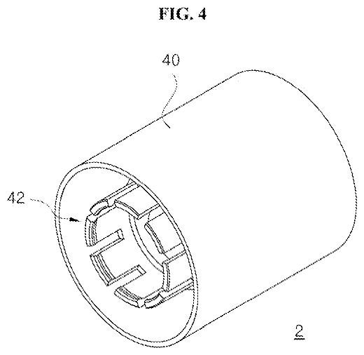

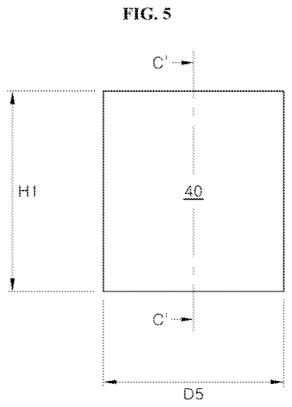

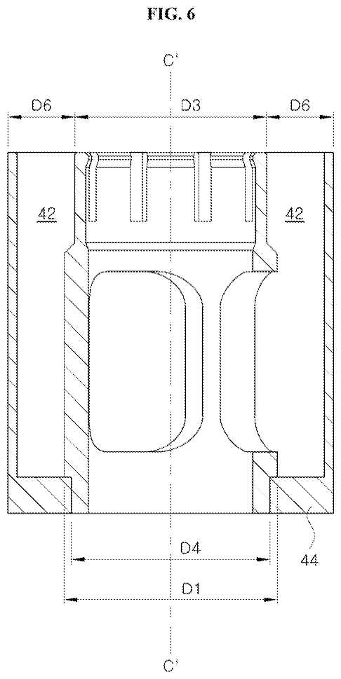

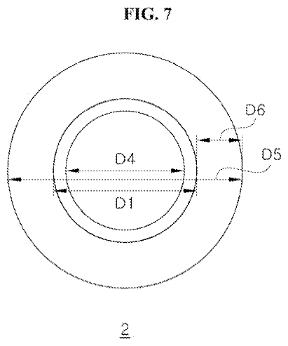

FIG. 4 is a lateral view for describing a dental implant surgical drill guide device according to another embodiment of the present disclosure, FIG. 5 is a cross-sectional view for describing the dental implant surgical drill guide device illustrated in FIG. 4, FIG. 6 is a view illustrating a cross-section along line C′-C′ illustrated in FIG. 5, and FIG. 7 is a bottom view for describing the dental implant surgical drill guide device illustrated in FIG. 5.

Referring to FIG. 4, a dental implant surgical drill guide device 2 according to another embodiment of the present disclosure may include a guard 40.

Except for the guard 40 illustrated in FIG. 4, the dental implant surgical drill guide device 2 may have the same characteristics as the dental implant surgical drill guide device 1 illustrated in FIGS. 1 to 3.

In the following description with reference to FIG. 4, detailed description of content overlapping the content described above with reference to FIGS. 1 to 3 may be omitted, and differences therefrom may be mainly described. Therefore, elements of the dental implant surgical drill guide device 2 illustrated in FIG. 4 that perform the same functions as the corresponding elements of the dental implant surgical drill guide device 1 illustrated in FIGS. 1 to 3 will be denoted by the same reference numerals, and detailed description thereof will be omitted.

First, referring to FIG. 4, the dental implant surgical drill guide device 2 may include the guard 40.

The guard 40 may be formed in a cylindrical shape having a predetermined diameter and extend in the longitudinal direction, and an empty space may be formed at an inner side surface of the guard 40.

In the present embodiment, the guard 40 may be made of the same material as the dental implant surgical drill guide device 1, for example, a material such as titanium or a titanium alloy that has excellent corrosion resistance and is stable even in human tissue, but the present disclosure is not limited thereto.

Specifically, the guard 40 may have a length corresponding to the length of the dental implant surgical drill guide device 1 and be disposed to be spaced a predetermined distance apart from the dental implant surgical drill guide device 1 to surround an outer side surface of the dental implant surgical drill guide device 1.

The guard 40 having an empty space formed therein may be formed to be spaced a predetermined distance apart from outer side surfaces of the body 10 and the neck 20 and coupled to an outer side surface of the extension 30. That is, the guard 40 may have a space 42 formed between the guard 40 and the body 10 and the guard 40 and the neck 20. Accordingly, washing water may be guided by the guard 40, move through the space 42, pass through the through-holes 12 of the body 10, and be smoothly delivered to a drilling site. That is, the guard 40 may guide an inflow and outflow of the washing water.

For example, in the cross-sectional views of FIGS. 5 and 6, in the case in which an overall height H1 of the dental implant surgical drill guide device 1 is 10 mm in the longitudinal direction, a height H1 of the guard 40 may be 10 mm, and a diameter D5 of the guard 40 may be 7.5 mm. Accordingly, a portion of the space 42 that corresponds to the neck 20 may have a diameter D6 of 1.1 mm, and a portion of the space 42 that corresponds to the body 10 may have a diameter D6 of 1 mm.

By the guard 40 having the space 42 provided as described above, the guard 40 may guide washing water introduced through the space 42 and prevent the washing water from being delivered to a site other than the drilling site to facilitate the inflow of the washing water and block the outflow of the washing water. In this way, necrosis of the alveolar bone 52 due to a temperature rise during the drilling process can be prevented.

Also, the guard 40 may have a catching portion 44 provided at a lower surface.

The catching portion 44 may be formed to protrude a predetermined thickness from an end of the guard 40 toward an inner side thereof. Here, a width of the space 42 having the catching portion 44 provided therein may be formed to be the same as the diameter D4 of the extension 30.

For example, in the case in which the height H2 of the body 10 is 6.5 mm, the diameter D1 of the body 10 is 5.9 mm, the height H4 of the neck 20 is 2.5 mm, the diameter D3 of the neck 20 is 5.3 mm, the height H6 of the extension 30 is 1 mm, and the diameter D4 of the extension 30 is 5.5 mm when the height H1 of the dental implant surgical drill guide device 1 is 10 mm in the longitudinal direction, a width of the catching portion 44 may be 1 mm.

By the catching portion 44 that protrudes toward the inner side of the guard 40 being coupled to the extension 30 formed to have a step with the outer side surface of the body 10, the dental implant surgical drill 50 can be further prevented from being detached from the body 10.

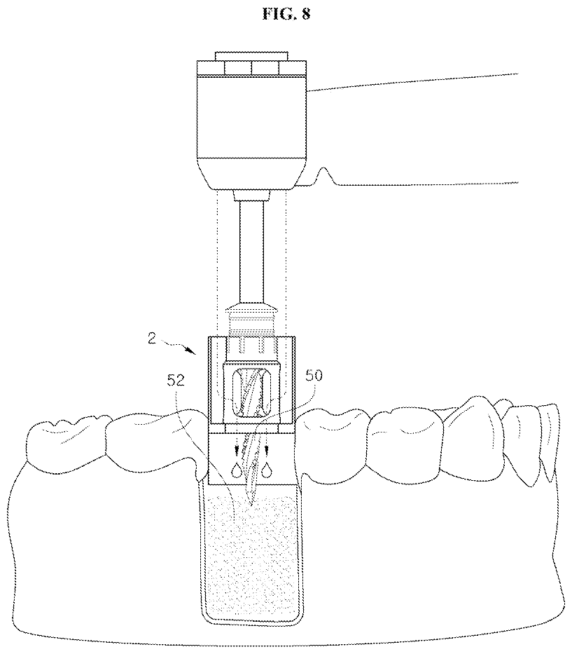

The operation of the dental implant surgical drill guide device according to another embodiment of the present disclosure that has the above-described structure is as follows. FIG. 8 is a view for describing a coupling state between the dental implant surgical drill guide device illustrated in FIG. 4 and a dental implant surgical drill.

Referring to FIG. 8, the dental implant surgical drill 50 and the dental implant surgical drill guide device 2 may be coupled to perform a drilling task.

Specifically, in the case in which the dental implant surgical drill 50 is rotated forward and fastened to the inside of the body 10, with the rotation of the dental implant surgical drill 50, the cutting portions 24 of the neck 20 are closed due to the cutting grooves 22, and the dental implant surgical drill 50 can be primarily fixed to the dental implant surgical drill guide device 2 due to the neck 20 and secondarily fixed to the dental implant surgical drill guide device 2 by the extension 30 having the diameter D4 smaller than the diameter D1 of the body 10. That is, since the dental implant surgical drill 50 can be further prevented from being detached from the body 10 by the catching portion 44 that protrudes toward the inner side of the guard 40 being coupled to the extension 30 formed to have a step with the outer side surface of the body 10, the drilling task can be stably performed for both a practitioner and a patient.

Next, for implant surgery, the dental implant surgical drill 50 coupled to the dental implant surgical drill guide device 2 may be moved downward toward a position above the corresponding alveolar bone 52 to drill a hole in the alveolar bone 52. Here, washing water may be supplied to cool the dental implant surgical drill 50 that becomes hot due to friction with the drilling site.

By the guard 40 of the dental implant surgical drill guide device 2 that guides the supplied washing water, the washing water rapidly moves through the space 42, passes through the through-holes 12, and is smoothly supplied to the drilling site without being delivered to the outside. In this way, necrosis of the alveolar bone 52 can be prevented, and the drilling task can be safely performed. Here, the washing water supplied to the drilling site through the space 42 of the guard 40 and the through-holes 12 may also serve as cooling water that cools the drilling site to prevent an excessive increase in the temperature of the drilling site due to frictional heat in the drilling process, and in this way, the washing water can, while preventing necrosis of the alveolar bone, allow foreign matter generated in the drilling process, for example, tissue in an oral cavity or fragments of the alveolar bone, to be smoothly discharged from the drilling site through the through-holes 12.

The embodiments of the present disclosure have been described above with reference to the accompanying drawings, but those of ordinary skill in the art to which the present disclosure pertains should understand that the present disclosure may be carried out in other specific forms without changing the technical spirit or essential features thereof. Therefore, the embodiments described above should be understood as illustrative, instead of limiting, in all aspects.

1. A dental implant surgical drill guide device comprising:
a body formed in a cylindrical shape having a first diameter and extending in a longitudinal direction;
a neck having a second diameter and formed to extend in the longitudinal direction from one side end of the body; and
an extension having a third diameter, formed to extend in the longitudinal direction from the other side end of the body, and having a stepped structure with the body,
wherein the body, the neck, and the extension are integrally formed,
a size of the third diameter is formed to be smaller than a size of the first diameter and larger than a size of the second diameter,
the dental implant surgical drill guide device further includes a guard which is formed in a cylindrical shape having a fourth diameter formed to be larger than each of the first to third diameters, which is formed to extend in the longitudinal direction and surround the body, the neck, and the extension from outer side surfaces thereof, which has an empty space formed at an inner side surface, and which is configured to guide an inflow and outflow of washing water,
the guard includes a space that is spaced a predetermined distance apart from the outer side surfaces of the body and the neck and a catching portion that is disposed at a lower end of the guard, protrudes from the lower end to the inner side surface, and is fitted and coupled to the stepped structure of the extension,
the space includes a first space formed between an outer side of the body and the inner side surface of the guard and having an upper portion formed to be exposed and a second space formed between an outer side of the neck and the inner side surface of the guard and having a lower portion blocked due to the catching portion being fitted and coupled to the extension,
a width of the first space is formed to be larger than a width of the second space, and
a width of the catching portion is formed to be the same as a width of the extension.

2. The dental implant surgical drill guide device of claim 1, wherein:
the body has one or more through-holes formed to have a predetermined shape and pass through a side surface of the body in a radial direction of the body; and
the neck includes cutting grooves formed to be opened by cutting the neck in the longitudinal direction and cutting portions formed in plate shapes having the same width due to the cutting grooves and formed alternately with the cutting grooves.

3. The dental implant surgical drill guide device of claim 1, wherein:
the dental implant surgical drill guide device is made of titanium and a titanium alloy;
the dental implant surgical drill guide device further comprises a connector formed between the body and the neck and having a predetermined length; and
the connector is formed to be inclined downward from the neck to the body.

4. A dental implant surgical drill guide device in which a dental implant surgical drill and the dental implant surgical drill guide device are combined to drill a hole in an alveolar bone and form a drilling site, the dental implant surgical drill guide device comprising:
the dental implant surgical drill guide device including a body which is formed in a cylindrical shape having a first diameter, extends in a longitudinal direction, and has one or more through-holes formed therein, a neck which has a second diameter smaller than the first diameter and is formed to extend in the longitudinal direction from one side end of the body, and an extension which has a third diameter smaller than the first diameter and larger than the second diameter and is formed to extend in the longitudinal direction from the other side end of the body; and
a guard configured to surround an outer side surface of the dental implant surgical drill guide device and have an empty space formed therein,
wherein the guard includes a first space formed between an outer side surface of the body and an inner side surface of the guard, a second space formed between an outer side surface of the neck and the inner side surface of the guard, and a catching portion coupled to the extension formed to have a step with the outer side surface of the body,
a width of the first space is formed to be larger than a width of the second space,
by gravity, washing water is delivered to the drilling site by moving to the first space and the second space and passing through the through-holes while leakage of the washing water is prevented by the guard.
